(19) United States
(12) Patent Application Publication
(10) Pub. No.: US 2007/0244213 A1
(43) Pub. Date: Oct. 18, 2007

(54) Silicone Emulsions and Their Preparation

(76) Inventor: Frank William Wallace, Brussels (BE)

Correspondence Address: Alan Zombeck;Dow Corning Corporation, 2200 W Salzburg Road, Midland, MI, 48686-0994, US

(21) Appl. No.: 11/632,194

(22) PCT Filed: Jul. 18, 2005
(86) PCT No.: PCT/US05/25442

§ 371 Date: Jan. 17, 2007

(30) Foreign Application Priority Data
Jul. 29, 2004 (GB) 0416890.2

(51) Int. Cl. C08J 3/03; A61K 8/06; C08L 83/04

(52) U.S. Cl. 523/102; 523/105; 523/122; 524/395; 524/588

(57) ABSTRACT

Multiple emulsions of the water-in-oil-in-water (W/O/W) type and processes for their preparation are disclosed. The multiple emulsions contain an oil phase comprising a mixture of: (i) a polysiloxane (S) which is a chain extension reaction product of a polysiloxane (S1) having reactive end groups, and (ii) a hydrophobic material (H) which is miscible with polysiloxane (S1) and with polysiloxane (S). The multiple emulsions allows for the entrapment and controlled delivery and release of water-soluble active ingredients.

### FIELD OF THE INVENTION

This invention relates to multiple emulsions of the water-in-oil-in-water (W/O/W) type and to processes for their preparation. The invention allows for the entrapment and controlled delivery and release of water-soluble active ingredients.

Water-soluble active ingredients, such as a fragrance, cleaning agent, hair conditioner, sunscreen, deodorant, vitamin, medication, biocide, dye, pest repellent, or catalyst, for use for example in personal care, cosmetic, or health care applications, or in industrial applications, may need to be protected against premature release of the active ingredient. Release may not be required until the composition has been topically applied or applied to a substrate.

### BACKGROUND TO THE INVENTION

U.S. Pat. No. 5,656,280 describes a W/O/W emulsion comprising an external aqueous phase containing a surfactant system capable of forming liquid crystals, and a primary emulsion comprising an internal aqueous phase containing a topically active compound, an oil phase comprising a volatile silicone or hydrocarbon compound, and a surfactant which is oil-soluble or has a HLB (hydrophobic lipophilic balance) value of 10 or less.

U.S. Pat. No. 5,948,855 describes a W/O/W emulsion in which an elastomeric silicone polyether is used to form a primary water-in-oil emulsion which is dispersed into the final aqueous continuous phase. U.S. Pat. No. 6,080,394 describes a similar multiple emulsion in which the internal phase is a non-aqueous polar solvent instead of water.

U.S. Pat. No. 6,013,682 describes a method of making a silicone in water emulsion by mixing at least one polysiloxane having reactive end groups, at least one organosilicon material that reacts with said polysiloxane by a chain extension reaction and a catalyst for said chain extension reaction to form a composition (I), and then mixing composition (I) with at least one surfactant and (III) water, and emulsifying the mixture. U.S. Pat. No. 6,013,682 suggests delivery of actives but through direct incorporation of active ingredients in the water or oil phase. U.S. Pat. No. 6,013,682 does not mention W/O/W emulsions.

### SUMMARY OF THE INTENTION

A water-in-oil-in-water emulsion according to the invention comprises an emulsion in an aqueous phase (A) of an oil phase (Y) which comprises a mixture of:
(i) a polysiloxane (S) which is a chain extension reaction product of a polysiloxane (S1) having reactive end groups, and
(ii) a hydrophobic material (H) which is miscible with polysiloxane (S1) and with polysiloxane (S) and has an aqueous or polar phase (A1) emulsified therein containing an active ingredient soluble in the aqueous or polar phase.

A process according to the invention for the preparation of a water-in-oil-in-water emulsion in which an emulsion (E1) of an aqueous or polar phase (A1) containing a dissolved active ingredient in a hydrophobic material (H) is dispersed in a continuous aqueous phase (A), characterized in that the emulsion (E1) is mixed with a polysiloxane (S1) having reactive end groups and chain extension of the polysiloxane (S1) is effected in the presence of the emulsion (E1), the polysiloxane (S1) being emulsified in the aqueous phase (A) during or before the chain extension reaction.

### DETAILED DESCRIPTION OF THE INVENTION

A water-in-oil emulsion is initially prepared by emulsifying an aqueous or polar phase (A1) in a suitable hydrophobic material (oil phase) (H) using a lipophilic surfactant. The lipophilic surfactant is generally a surfactant of low HLB (hydrophilic-lipophilic balance), for example a HLB of 1-10. The active ingredient may be dissolved in the aqueous or polar phase (A1) prior to emulsification.

The active ingredient can for example be a fragrance composition. The fragrance composition may be solid or liquid and may be a single fragrant compound or a mixture. The fragrance composition may be a perfume for incorporation in a personal care product such as a skin cream, shampoo or face cream or in a cleaning composition for household use, or may be a flavour or aroma compound to be applied for example to food or food packaging. Alternative active ingredients include sunscreen materials, vitamins, for example Vitamin C or a water-soluble derivative thereof biocides, pest and insect repellents and pharmaceutically active materials.

The aqueous or polar phase (A1) is usually an aqueous solution of the active ingredient but can be a solution in a mixture of water and a water-miscible polar solvent such as an alcohol, or can be a solution in a polar organic solvent provided that this is immiscible with the oil (H).

The oil (H) is preferably a silicone oil. The silicone oil is preferably of low viscosity, generally below 1000 mPa·s and most preferably below 500 mPa·s, for example in the range 0.1 to 100 mPa·s. The silicone oil is preferably a linear polydiorganosiloxane but can contain some branching. The polydiorganosiloxane (C) is preferably polydimethylsiloxane, although it can contain other lower alkyl groups, for example ethyl. The polydimethylsiloxane preferably has trimethylsilyl terminal units, but alternative terminal units can be present in the polydimethylsiloxane, for example silanol groups. The oil (H) can alternatively be an organic material, particularly a hydrocarbon such as a mineral oil, provided that it is miscible with polysiloxane (S1) and with polysiloxane (S).

Examples of suitable lipophilic surfactants which can be used to form the emulsion (E1) include silicone polyether surfactants.

The water-in-oil emulsion (E1) can be prepared using a direct process including high-pressure emulsification equipment such as a homogeniser or sonolator. It can alternatively be prepared using a phase inversion or a thick phase process, in which the emulsion is made at a high water to oil phase ratio and sheared to small particle size using a change-can type mixer or a rotor/stator type mixer. The particle size of the internal water droplets in E1 must be significantly smaller than the desired oil droplet size in the final WOW emulsion. The particle size of the internal water droplets in E1 can for example be in the range 0.1 to 10 μm.

The emulsion (E1) is mixed in the desired ratio with a polysiloxane (S1) having reactive end groups, for example in a ratio of 1:3 to 10:1. A high ratio of (E1) to (S1) is usually preferred to give a higher concentration of the active ingredient. A chain extension reaction of the polysiloxane (S1) is then effected in the presence of the emulsion (E1). The emulsion (E1) is preferably mixed with a polysiloxane (S1) having reactive end groups, at least one organosilicon material that reacts with said polysiloxane (S1) by a chain extension reaction and/or a catalyst for said chain extension reaction, and the resulting composition is mixed with at least one surfactant and water, and emulsified. Two alternative chemistries which may be used for the chain extension reaction are hydrosilylation or silanol-silanol condensation.

If hydrosilylation is used, the reactive end groups of the polysiloxane (S1) are aliphatically unsaturated groups, the organosilicon material is a polysiloxane having at least one Si—H group, and the catalyst is a platinum or rhodium containing catalyst. The polysiloxane (S1) is generally a substantially linear polydiorganosiloxane and preferably has the structure: 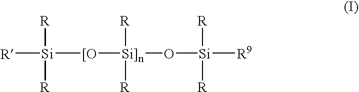
where R represents a hydrocarbon group having up to 20 carbon atoms such as an alkyl (e.g., methyl, ethyl, propyl or butyl), or aryl (e.g., phenyl) group and R′ represents the aliphatically unsaturated group required for the chain extension reaction, for example vinyl, allyl or hexenyl; and n is an integer greater than 1. Preferably there is on average between one and two reactive groups (inclusive) per polymer, most preferably two groups or just less. Preferably, a majority, more preferably over 90%, and most preferably over 98% of the reactive groups are end-groups R′ as shown. Preferably n is an integer such that the polydiorganosiloxane has a viscosity between 1 and 1×10⁶ mm²/sec at 25° C. If desired, the polydiorganosiloxane can have a small amount of branching (e.g., less than 2 mole % of the siloxane units) without affecting the invention, i.e., the polymers are ‘substantially linear’. The groups R are usually hydrocarbyl groups, for example alkyl or aryl groups; preferably at least 80% of the R groups are alkyl groups, more preferably methyl groups. If desired, R groups can be substituted with, for instance, oxygen containing groups such as epoxy or alcohol groups.

The organosilicon material having at least one Si—H group preferably has the above structure (I) wherein R, R′ and n are as defined above and provided that on average between one and two (inclusive) R or R′ groups comprise hydrogen atoms and n is 0 or a positive integer. Preferably the Si—H groups are terminal groups R′. This material can be a polymer or a lower molecular weight material such as a disiloxane or trisiloxane.

The catalyst may tale the form of platinum or rhodium deposited on a carrier such as silica gel or powdered charcoal, or a platinum or rhodium salt or compound such as platinic chloride or chloroplatinic acid or a platinum or rhodium complex. Catalysts comprising Pt^{IV}, for example platinic chloride or chloroplatinic acid, or a complex prepared from chloroplatinic acid hexahydrate and divinyltetramethyldisiloxane, are particularly preferred. Generally, the catalyst is used at between 0.0001 and 10 wt. % based on the weight of the polysiloxane (S1).

Hydrosilylation has the advantage that it can produce very high molecular weight polymer by reaction at room temperature, under neutral pH, and with a variety of surfactants; this can be useful for encapsulating sensitive or relatively unstable active ingredients. The process can produce emulsions in which the mean particle size is in the range of about 0.3 μm, for example 1 to 100 μm, and the viscosity of the silicone (S) is greater than 100 Pa·s., for example 1000 to 100000 Pa·s. The surfactant present during hydrosilylation can in general be a non-ionic surfactant, a cationic surfactant, an anionic surfactant or an amphoteric surfactant, although not all procedures for carrying out the process of the invention can be used with all surfactants. The amount of surfactant used will vary depending on the surfactant, but generally is between 1 and 30 wt. % based on the polydiorganosiloxane.

Examples of non-ionic surfactants include polyoxyalkylene alkyl ethers such as polyethylene glycol long chain (12-14C) alkyl ether, polyoxyalkylene sorbitan ethers, polyoxyalkylene alkoxylate esters, polyoxyalkylene alkylphenol ethers, ethylene glycol propylene glycol copolymers and alkylpolysaccharides.

Examples of cationic surfactants include quaternary ammonium hydroxides such as octyl trimethyl ammonium hydroxide, dodecyl trimethyl ammonium hydroxide, hexadecyl trimethyl ammonium hydroxide, octyl dimethyl benzyl ammonium hydroxide, decyl dimethyl benzyl ammonium hydroxide, didodecyl dimethyl ammonium hydroxide, dioctadecyl dimethyl ammonium hydroxide, tallow trimethyl ammonium hydroxide and coco trimethyl ammonium hydroxide as well as corresponding salts of these materials, fatty amines and fatty acid amides and their derivatives, basic pyridinium compounds, quaternary ammonium bases of benzimidazolines and polypropanolpolyethanol amines.

Examples of suitable anionic surfactants include alkyl sulfates such as lauryl sulfate, alkylbenzenesulfonic acids and salts; the sulfate esters of monoalkyl polyoxyethylene ethers; alkylnapthylsulfonic acid; alkali metal sulforecinates, sulfonated glyceryl esters of fatty acids such as sulfonated monoglycerides of coconut oil acids, salts of sulfonated monovalent alcohol esters, amides of amino sulfonic acids, sulfonated products of fatty acid nitrites and condensation products of naphthalene sulfonic acids with formaldehyde.

Examples of suitable amphoteric surfactants include cocamidopropyl betaine, cocamidopropyl hydroxysulfate, cocobetaine, sodium cocoamidoacetate, cocodimethyl betaine, N-coco-3-aminobutyric acid and imidazolinium carboxyl compounds.

In one preferred process according to the invention, the polysiloxane (S1) having aliphatically unsaturated groups, the emulsion (E1), the organosilicon material having at least one Si—H group, the catalyst and the surfactant are mixed and are emulsified in water. The polysiloxane (S1), the organosilicon material, the catalyst and the surfactant can be mixed all at once or these materials can be mixed in any order. However when the polydiorganosiloxane, the organic material and the catalyst are combined, the polymerisation reaction begins. As such, it may be preferred to mix one of these components of the composition last. For example, it may be preferred to premix the metal containing catalyst, the organosilicon material and the surfactant before mixing with the polysiloxane (S1) and the emulsion (E1). Alternatively the polysiloxane (S1), the emulsion (E1), the organosilicon material and the surfactant can be premixed before mixing with the catalyst.

It is preferred that all the above materials are mixed before emulsifying in water, so that all the materials required for polymerisation are mixed before the composition is emulsified and polymerisation commences before the composition is emulsified, as described in U.S. Pat. No. 6,013,682. Chain extension polymerisation of the polysiloxane (S1) then takes place at the interior of the oil droplets of polysiloxane (S1) in the emulsion. This allows for the production of silicone emulsions with independent control of particle size and of the silicone phase viscosity. The particle size is controlled by the amount of surfactant and water and the degree of mechanical shear applied to form the emulsion. The degree of polymerisation, and hence viscosity, is not controlled by droplet size, but by the ratio of materials used in the chain extension, in particular by the molar proportion of reagents and the ratio of catalyst to reagents.

The materials can alternatively be emulsified in water before addition of catalyst so that chain expansion takes place under emulsion polymerisation conditions.

For chain extension by silanol-silanol condensation, the reactive end groups of the polysiloxane (S1) are Si—OH groups and the chain extension of the polysiloxane (S1) is preferably effected in the presence of a surface-active acid catalyst. The polysiloxane (S1) is preferably a substantially linear polydiorganosiloxane. The organo groups in each siloxane unit are usually hydrocarbyl groups, for example alkyl or aryl groups; preferably at least 80% of the organo groups are alkyl groups, more preferably methyl groups. The degree of polymerisation of S1 can vary from 2-300, thus providing oligomers having a viscosity at 25° C. ranging from about 20 mPa·s to about 100 Pa·s.

The surface-active acid catalyst is an anionic surfactant having free acid groups, for example sulphonic acid groups. Examples of surface-active acid catalysts are alkyl or dialkylbenzelesulfonic acids such as hexylbenzenesulfonic acid, octylbenzenesulfonic acid, decylbenzenesulfonic acid, dodecylbenzenesulfonic acid, cetylbenzenesulfonic acid and myristylbenzenesulfonic acid; the sulfate esters of monoalkyl polyoxyethylene ethers; alkyl or dialkyl napthalene sulfonic acids; alkali metal sulforecinates, sulfonated glyceryl esters of fatty acids such as sulfonated monoglycerides of coconut oil acids, sulfonated monovalent alcohol esters or sulfonated products of fatty acid nitrites. Dialkyl benzene sulfonic acids or dialkyl naphthalene sulfonic acids as described in U.S. Pat. No. 6,235,834 may be particularly preferred as leading to emulsions of large particle size such as 1-100 μm; examples are di(n-propyl)benzene sulfonic acid, di(tert-butyl) benzene sulfonic acid, dihexyl benzene sulfonic acid, dioctyl benzene sulfonic acid, dinonyl benzene sulfonic acid, didodecyl benzene sulfonic acid, distearyl benzene sulfonic acid, ditetradecyl benzene sulfonic acid, dihexadecyl benzene sulfonic acid, dioctadecyl benzene sulfonic acid, di(2-ethylhexyl) benzene sulfonic acid, di(2-butyloctyl) benzene sulfonic acid, di(2-amylnonyl) benzene sulfonic acid, di(n-propylheptyl) benzene sulfonic acid, di(n-propyl) naphthalene sulfonic acid, di(iso-propyl) naphthalene sulfonic acid, di(sec-butyl ) naphthalene benzene sulfonic acid, dihexyl naphthalene sulfonic acid, dioctyl naphthalene sulfonic acid, dinonyl naphthalene sulfonic acid,didodecyl naphthalene sulfonic acid, distearyl naphthalene sulfonic acid, ditetradecyl naphthalene sulfonic acid, dihexadecyl naphthalene sulfonic acid, di(2-ethylhexyl) naphthalene sulfonic acid, and di(2-ethyloctyl) naphthalene benzene sulfonic acid. Commercial products representative of such catalysts are SYNEX® DN-052 and NACURE® 1052, both trademarks of King Industries, Norwalk, Conn. for the product dinonyl (C₉) naphthalene sulfonic acid; ARISTONIC Acid VH, a mixture of unspecified monoalkyl and dialkyl benzene sulfonic acids, and ARISTONIC ACID E, a C₁₂ dialkyl benzene sulfonic acid, both products of Pilot Chemical Company, Santa Fe Springs, Calif.

The surface-active acid catalyst is preferably used at 0.5 to 50%, preferably 1 to 20%, by weight based on the polysiloxane (S1), and at 0.05-25 percent by weight of the total emulsion, preferably 0.5-20 percent. The surface-active acid catalyst can be used as the only surfactant in the emulsion, but is preferably used in conjunction with a nonionic or anionic surfactant, which can for example be selected from those listed above. The nonionic or anionic surfactant is preferably present at 0.1-40 percent by weight of the total emulsion, most preferably 0.5-30 percent by weight.

The polysiloxane (S1), emulsion (E1) and the surface active acid catalyst can be mixed before emulsification in water, in which case polymerisation commences before the composition is emulsified and chain extension polymerisation of the polysiloxane (S1) then takes place at the interior of the oil droplets of polysiloxane (S1) in the emulsion. In this procedure the non-ionic surfactant can be premixed with the polysiloxane (S1), emulsion (E1) and surface active acid catalyst or can be mixed with the water into which the polysiloxane (S1) is emulsified. Alternatively the polysiloxane (S1) and emulsion (E1) can be contacted with the surface active acid catalyst and water simultaneously, or the polysiloxane (S1) can be emulsified with a nonionic or anionic surfactant and then contacted with the surface active acid catalyst, so that chain extension proceeds by emulsion polymerisation. If the polysiloxane (S1) is thus pre-emulsified, emulsification may be by a direct process including high-pressure emulsification equipment or by a phase inversion or thick phase process, in which the emulsion is made at a high oil to water phase ratio and sheared to small particle size. The emulsion (E1) is preferably mixed with the polysiloxane (S1) after any high-pressure or high-shear emulsification step, to ensure that the aqueous phase (A) is not released from the emulsion (E1), but before the polysiloxane (1) contacts the surface active acid catalyst.

When the desired degree of polymerisation has taken place, which may for example be in the range 30 minutes to 5 days at ambient temperature or less at higher temperatures, polymerisation can be stopped by neutralization of the surface active acid catalyst. Neutralisation is preferably by an amine, most preferably a tertiary amine such as triethanolamine.

Chain extension of polysiloxane (S1) by silanol-silanol condensation has the advantage that it is economical because the reagents are less expensive that those used in hydrosilylation, and polymer viscosity can be controlled in the range of 65 mPa·s to 1500 Pa·s. Polymerisation under the conditions described in U.S. Pat. No. 6,235,834 is especially suited to the production of large particle size emulsions, which are most practical for W/O/W systems.

The silicone W/O/W emulsions of the present invention are useful as a means for topical delivery of actives. The process of the present invention involving a chain extension reaction, especially the suspension polymerisation methods described, allow for control of the viscosity of the silicone polymer matrix (S). The polymer matrix viscosity plays a large part in control of actives delivery in the final topical application.

The invention is illustrated by the following Examples, in which % are percentages by weight.

### EXAMPLE 1

0.9% Dow Corning (Trade Mark) 2-5185C silicone polyether surfactant was added to 17% 0.65 cst (about 0.65 mPa·s) trimethylsilyl-terminated polydimethylsiloxane. 49% saltwater (water with dissolved NaCl serving to simulate an active ingredient) was slowly added to the polydimethylsiloxane mixture with high speed mixing to form an emulsion thick phase. The thick phase was diluted with an additional 33% 0.65 cst polydimethylsiloxane to form a water-in-oil emulsion (E1).

60% dimethylvinylsiloxy terminated polydimethylsiloxane was mixed with 1.9% hydrogen terminated polydimethylsiloxane. 3% of the emulsion (E1) was added and mixed in. 0.03% Dow Coming 2-0707 (platinum catalyst) was added and mixed. 7.7% Arquad 16-29 (Trade Mark) cationic surfactant (hexadecyltri-methylamonium chloride) and 2.8% water were than added and mixed at high speed. A W/O/W emulsion was produced.

### EXAMPLE 2

1.2% Dow Corning 2-5135C was added to 16% Dow Cornilng Q1-3563 silanol-terminated linear polydimethylsiloxane and mixed. 49% saltwater was slowly added with high speed mixing to form an emulsion thick phase. 33% more Q1-3563 was added with mixing to form a water-in-oil emulsion (E1).

7.3% of this emulsion (E1) was added to 41% Q1-3563 and mixed. 48% water, 2.3% Nacure 1051 (dialkyl napthalene sulfonic acid), and 0.6% Renex 30 nonionic surfactant (polyoxyethylated C11-14 alcohol) were added and mixed by shaking. The emulsion was allowed to react for 16 hours at room temperature, then neutralize with 1.5% triethanolamine. The product was a W/O/W emulsion.

1. A water-in-oil-in-water emulsion comprising an emulsion in an aqueous phase (A) of an oil phase (Y) which comprises a mixture of:
(i) a polysiloxane (S) which is a chain extension reaction product of a polysiloxane (S1) having reactive end groups, and
(ii) a hydrophobic material (H) which is miscible with polysiloxane (S1) and with polysiloxane (S) and has an aqueous or polar phase (A1) emulsified therein containing an active ingredient soluble in the aqueous or polar phase.

2. An emulsion according to claim 1 wherein the mean particle size of the emulsified droplets of oil phase (Y) is at least 0.3 μm.

3. An emulsion according to claim 1 wherein the hydrophobic material (H) is a non-reactive silicone fluid.

4. An emulsion according to claim 1 wherein the viscosity of the hydrophobic material (H) is 0.1-500 mPa·s.

5. An emulsion according to claim 1 wherein the viscosity of the polysiloxane (S) is at least 10⁵ mPa·s.

6. An emulsion according to claim 1 wherein the water soluble active ingredient is a fragrance, cleaning agent, hair conditioner, sunscreen, deodorant, vitamin, medication, biocide, dye, pest repellent, or catalyst.

7. A process for the preparation of a water-in-oil-in-water emulsion in which an emulsion (E1) of an aqueous or polar phase (A1) containing a dissolved active ingredient in a hydrophobic material (H) is dispersed in a continuous aqueous phase (A), wherein the emulsion (E1) is mixed with a polysiloxane (S1) having reactive end groups and chain extension of the polysiloxane (S1) is effected in the presence of the emulsion (E1), the polysiloxane (S1) being emulsified in the aqueous phase (A) during or before the chain extension reaction.

8. A process according to claim 7, wherein the emulsion (E1) is mixed with a polysiloxane (S1) having reactive end groups, at least one organosilicon material that reacts with said polysiloxane (S1) by a chain extension reaction and a catalyst for said chain extension reaction, and the resulting composition is mixed with at least one surfactant and water, and emulsified.

9. A process according to claim 8, wherein the reactive end groups of the polysiloxane (S1) are aliphatically unsaturated groups, the organosilicon material is a polysiloxane having at least one Si—H group and the catalyst is a platinum or rhodium containing catalyst.

10. A process according to claim 7, wherein the reactive end groups of the polysiloxane (S1) are Si—OH groups and the chain extension of the polysiloxane (S1) is effected in the presence of a surface-active acid catalyst.

11. A process according to claim 7, wherein the surface-active acid catalyst is a dialkyl benzene sulfonic acid or dialkyl naphthalene sulfonic acid.
